(19) United States
(12) Patent Application Publication
(10) Pub. No.: US 2011/0152610 A1
(43) Pub. Date: Jun. 23, 2011

(54) INTRALUMENAL ACCESSORY TIP FOR ENDOSCOPIC SHEATH ARRANGEMENTS

(75) Inventors: Robert M. Trusty, Cincinnati, OH (US); Omar J. Vakharia, Cincinnati, OH (US)

(73) Assignee: Ethicon Endo-Surgery, Inc., Cincinnati, OH (US)

(21) Appl. No.: 12/640,492

(22) Filed: Dec. 17, 2009

(51) Int. Cl. A61B 1/00

(52) U.S. Cl. 600/104

(57) ABSTRACT

An intralumenal accessory tip for use with an inner sheath assembly during initial insertion of the inner sheath into a patient. The intralumenal accessory tip is removably attachable to the distal end of the inner sheath assembly and has passages therein to accommodate access tubes and endoscopic tools protruding from the inner sheath assembly. The intralumenal accessory tip may be attached to the distal end of the inner sheath assembly and both assemblies may be inserted into an overtube to permit the inner sheath assembly to be inserted into the patient. The inner sheath assembly may then be withdrawn out of the overtube and the intralumenal tip accessory removed therefrom.

### BACKGROUND

The embodiments relate, in general, to endoscopes and medical procedures and, more particularly, to devices for facilitating the insertion and manipulation of endoscopic sheath assemblies and other surgical instruments within a body cavity to accomplish various surgical and therapeutic procedures.

Minimally invasive procedures are desirable because such procedures can reduce pain and provide relatively quick recovery times as compared with conventional open medical procedures. Many minimally invasive procedures are performed through one or more ports through the abdominal wall, commonly known as trocars. A laparascope that may or may not include a camera may be used through one of these ports for visualization of the anatomy and surgical instruments may be used simultaneously through other ports. Such devices and procedures permit a physician to position, manipulate, and view anatomy, surgical instruments and accessories inside the patient through a small access opening in the patient's body.

Still less invasive procedures include those that are performed through insertion of an endoscope through a natural body orifice to a treatment region. Examples of this approach include, but are not limited to, cystoscopy, hysteroscopy, esophagogastroduodenoscopy, and colonoscopy. Many of these procedures employ the use of a flexible endoscope and flexible or steerable sheath assemblies during the procedure. Flexible endoscopes often have a flexible, steerable articulating section near the distal end that can be controlled by the user utilizing controls at the proximal end. Treatment or diagnosis may be completed intralumenally, such as polypectomy or gastroscopy. Alternatively, treatment or diagnosis of extra-luminal anatomy in the abdominal cavity may be completed translumenally, for example, through a gastrotomy, colonotomy or culpotomy. Minimally invasive therapeutic procedures to treat or diagnose diseased tissue by introducing medical instruments translumenally to a tissue treatment region through a natural opening of the patient are known as Natural Orifice Translumenal Endoscopic Surgery (NOTES)™.

Regardless of the type of surgery involved and the method in which the endoscope is inserted into the body, the surgeons and surgical specialists performing such procedures have generally developed skill sets and approaches that rely on anatomical alignment for both visualization and tissue manipulation purposes. However, due to various limitations of those prior overtube and sheath arrangements, the surgeon may often times be forced to view the surgical site in such a way that is unnatural and thereby difficult to follow and translate directional movement within the operating theater to corresponding directional movement at the surgical site.

Over the years, a variety of different endoscope arrangements as well as various types of steerable sheaths and overtubes for accommodating endoscopes have been developed. For example, various endoscopic guide systems and endoscopes are disclosed in U.S. patent application Ser. No. 12/468,462, entitled “Manipulatable Guide System and Methods For Natural Orifice Translumenal Endoscopic Surgery”, filed May 19, 2009, the disclosure of which is herein incorporated by reference in its entirety. Some of the guide system embodiments disclosed therein include extended articulatable working channels as well as a liftable camera device. Such configurations afford the clinician with the ability to advantageously manipulate and position instruments passing through the working channels while providing the flexibility to position the camera to provide a “bird's eye”, “stadium”, or laparoscopic view of the theater. While such device represents a vast improvement over prior endoscope guide systems, the front face of the device does not afford the camera with much protection from organic material as the device is initially inserted into a patient's lumen or cavity. Moreover, the device does not particularly seek to be guided by and stay centered in the lumen as well as a more conventional endoscope. Also, the forward-protruding sheaths and endoscopic tools can undesirably contact and damage fragile tissue when the device is initially being inserted into the patient.

Consequently a need exists for a device that can protect the distal end of an inner sheath assembly and the sheaths and tools protruding therefrom when the device is initially inserted into a patient without the aid of a conventional overtube.

Still another need exists for a device that protects the camera lens from becoming fouled or obstructed with organic matter as the device and camera are being initially inserted into the patient or, while also forming access lumens that could also accommodate other endoscopic tools therethrough, such as, for example, a lens cleaning jet as well as other forms of endoscopic tools.

The foregoing discussion is intended only to illustrate some of the shortcomings present in the field at the time, and should not be taken as a disavowal of claim scope.

### SUMMARY

In one embodiment, an intralumenal accessory tip is provided for attachment to an inner sheath assembly that has a housing and at least one flexible access tube and an endoscopic tool protruding from a distal end thereof. In various embodiments, the intralumenal accessory tip may comprise a body portion that has a distal end and a proximal end. The proximal end may be configured for removable attachment to the housing. The intralumenal accessory tip may further include at least one tool-receiving passage that corresponds to the endoscopic tool. The tool-receiving passage may be sized to receive an endoscopic tool therein when the proximal end of the body portion is attached to the distal end of the housing such that no part of the endoscopic tool protrudes beyond the distal end of the body portion. The intralumenal accessory may further have a tube-receiving passage that is sized to receive at least one of the at least one flexible access tubes therein when the proximal end of the body portion is attached to the distal end of the housing such that no portion of the at least one flexible access tube protrudes beyond the distal end of said body portion.

In another general embodiment, an intralumenal accessory tip is provided for attachment to an inner sheath assembly. The inner sheath assembly may have a housing and two articulatable access tubes protruding therefrom as well as an endoscopic camera. The intralumenal accessory tip may further have a body portion that has a distal end and a proximal end. The intralumenal accessory tip may further include means for removably attaching the proximal end of said body portion to the distal end of the housing. In addition, the intralumenal accessory tip may include tube access means in the body portion for accommodating the two articulatable access tubes when the body portion is attached to the distal end of the housing. The intralumenal accessory tip may also include tool access means in the body portion for accommodating the endoscopic camera therein when said body portion is attached to the distal end of the housing.

In yet other general embodiments, a surgical method is disclosed. In various embodiments, the method may include attaching an intralumenal accessory tip onto a distal end of an internal sheath assembly such that all of any portions of accessory tubes and endoscopic tools protruding from the distal end of the internal sheath assembly are received within the intralumenal accessory tip and do not protrude beyond a distal end of the intralumenal accessory tip. The method may further include inserting the intralumenal accessory tip and a portion of internal sheath assembly into a patient such that the intralumenal accessory tip is located in a desired area. The method may also include inserting an overtube over the internal sheath assembly and the intralumenal accessory tip such that a distal end of the overtube is located in the desired area and a proximal end of the overtube protrudes out of the patient. The method includes withdrawing the internal sheath assembly and intralumenal accessory tip out through the proximal end of the overtube while retaining the overtube in place. The method further includes removing the intralumenal accessory tip from the distal end of the internal sheath assembly. In addition, the method may include reinserting the internal sheath assembly into the overtube to the desired area.

### BRIEF DESCRIPTION OF THE FIGURES

The novel features of the embodiments described herein are set forth with particularity in the appended claims. The embodiments, however, both as to organization and methods of operation may be better understood by reference to the following description, taken in conjunction with the accompanying drawings as follows.

FIG. 1 is a perspective view of an inner sheath assembly with which various intralumenal accessory tip embodiments of the present invention may be employed;

FIG. 2 is an exploded view of the inner sheath assembly of FIG. 1;

FIG. 3 is front perspective view of the housing of the inner sheath assembly of FIGS. 1 and 2;

FIG. 4 is a rear elevational view of the housing of FIG. 3;

FIG. 5 is a side view of the housing and first actuator of the inner sheath assembly of FIGS. 1-4;

FIG. 6 is a bottom view of a distal portion of the inner sheath assembly of FIGS. 1-5;

FIG. 7 is a perspective view of an intralumenal tip accessory embodiment of the present invention;

FIG. 8 is a front elevational view of the intralumenal tip accessory of FIG. 7;

FIG. 9 is a rear elevational view of the intralumenal tip accessory of FIGS. 7 and 8;

FIG. 10 is an exploded perspective view of the intralumenal tip accessory of FIGS. 7-9 oriented for attachment to an inner sheath assembly of the type depicted in FIGS. 1-6;

FIG. 11 is a side elevational view of the intralumenal tip accessory and inner sheath assembly of FIG. 10;

FIG. 12 is another side elevation view illustrating the intralumenal tip accessory coupled to the housing portion of the inner sheath assembly and oriented relative to a proximal end of a conventional overtube;

FIG. 13 is a diagrammatic view of an intralumenal tip accessory embodiment attached to an inner sheath assembly and inserted through the mouth of a patient;

FIG. 14 is another diagrammatic view of the inner sheath assembly reinserted through the overtube after the intralumenal accessory tip has been removed therefrom;

FIG. 15 is a front perspective view of another housing embodiment of an inner sheath assembly with which the various intralumenal accessory tip embodiments of the present invention may be used;

FIG. 16 is a rear perspective view of the housing of FIG. 15;

FIG. 17 is a rear elevational view of the housing of FIGS. 15 and 16; and

FIGS. 18, 19, and 20 illustrate engagement of the distal tip portion of an endoscopic instrument by a ramped guide surface of the housing of FIGS. 15-17.

### DETAILED DESCRIPTION

Certain embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting embodiments and that the scope of these embodiments is defined solely by the claims. The features illustrated or described in connection with one embodiment may be combined with the features of other embodiments. Such modifications and variations are intended to be included within the scope of the appended claims.

The various embodiments generally relate to guide systems and steerable sheath arrangements for use in connection with endoscopes for selectively positioning and manipulating endoscopic tools in a desired orientation within the body cavity. The term “endoscopic tools,” as used herein may comprise, for example, endoscopes, lights, insufflation devices, cleaning devices, suction devices, hole-forming devices, imaging devices, cameras, graspers, clip appliers, loops, Radio Frequency (RF) ablation devices, harmonic ablation devices, scissors, knives, suturing devices, etc. However, such term is not limited to those specific devices. As the present Description proceeds, those of ordinary skill in the art will appreciate that the unique and novel features of the various instruments and methods for use thereof may be effectively employed to perform surgical procedures by inserting such endoscopic tools through a natural body lumen (mouth, anus, vagina) or through a transcutaneous port (abdominal trocar, cardiothoracic port) to perform surgical procedures within a body cavity.

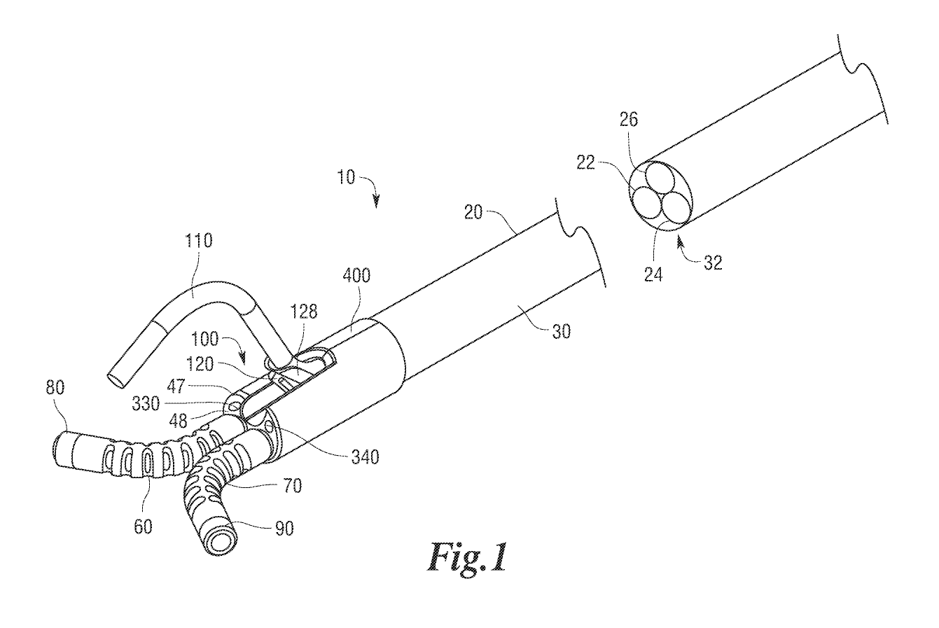

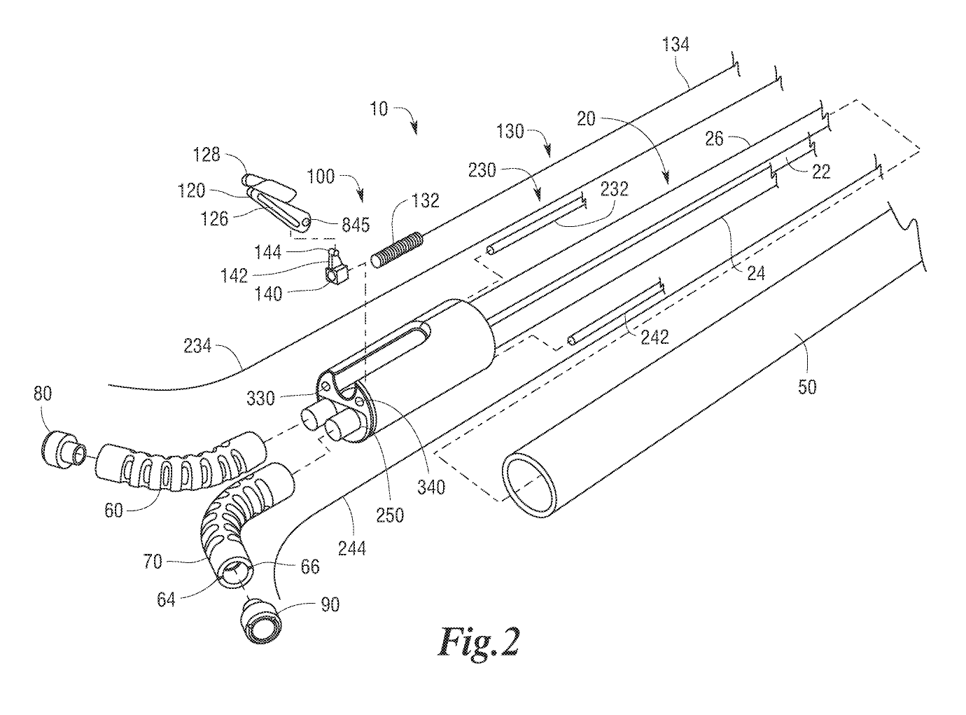

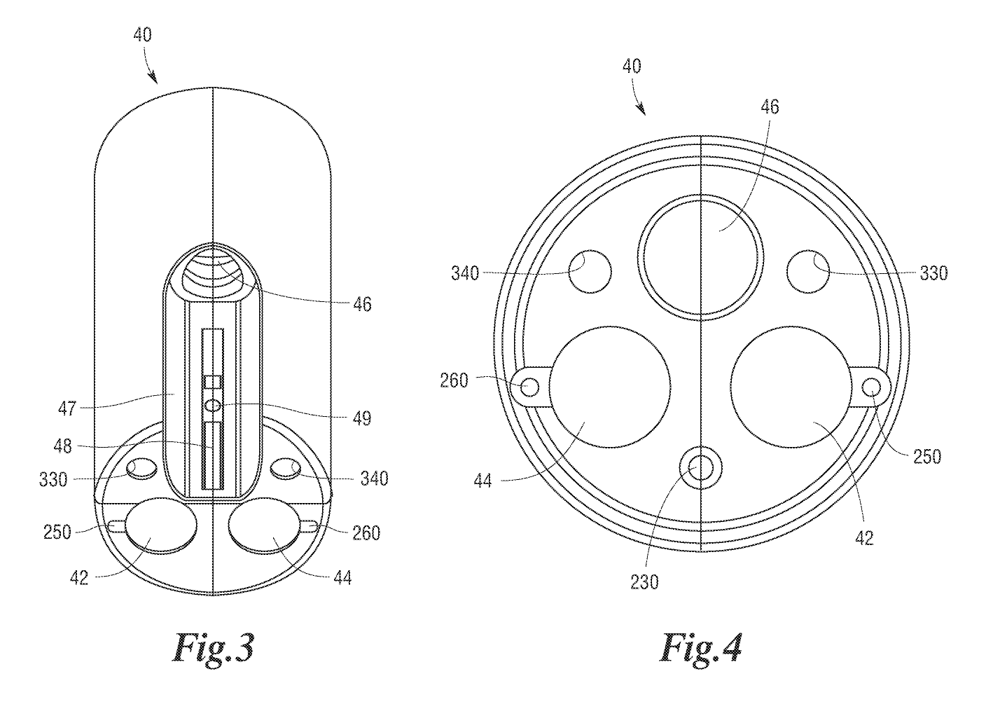

FIGS. 1 and 2 illustrate an assembled view and an exploded view, respectively, of an inner sheath assembly 10 of the type and construction disclosed in the aforementioned U.S. patent application Ser. No. 12/468,462, the disclosure of which has been herein incorporated by reference in its entirety. As shown, an embodiment of the inner sheath assembly 10 may comprise an inner sheath 20 that includes a plurality of working channels bundled over a common portion of their respective lengths by a flexible sleeve 30 to define a honeycombed cross-sectional area 32. Although the inner sheath 20 is depicted as comprising three working channels 22, 24, 26, it will be appreciated that the number of working channels may generally be two or more. The inner sheath assembly 10 may further comprise a housing 40 defining bores 42, 44, 46 (FIGS. 3 and 4) extending longitudinally and at least partially through the housing 40, with the bores 42, 44, 46 receiving distal ends of the working channels 22, 24, 26, respectively, at least partially therethrough. The distal ends of the working channels 22, 24 may be respectively received through the bores 42, 44 such that distal portions of the working channels 22, 24 coextend from a distal face of the housing 40, and the distal end of the working channel 26 may be received partially through the bore 46 and terminate within the housing 40 proximal the distal ends of the workings channels 22, 24. Articulation joints 60, 70 may respectively attach to distal ends of the working channels 22, 24, and distal tips 80, 90 may respectively attach to the distal ends of the articulation joints 60, 70. In certain embodiments and, as discussed in further detail below, the inner sheath assembly 10 may comprise a first actuator 100 to selectively position a distal end of an endoscopic tool 110 (e.g., camera, a light) introduced through the working channel 26 and/or one or more second actuators to manipulate the articulation joints 60, 70 such that distal ends of endoscopic tools introduced therethrough may be selectively positioned.

FIGS. 3 and 4 illustrate a front perspective view and a rear view, respectively, of the housing 40. The housing 40 may be fabricated from a suitable biocompatible metal or plastic, for example, and, in addition to bores 42, 44, 46, may define a recess 47 in communication with the bore 46 and generally aligned therewith. The recess 47 may be suitably dimensioned to receive and to guide a distal end of an endoscopic instrument introduced through the bore 46 via the working channel 26 and to accommodate components of the first actuator 100. As shown in FIG. 1, for example, the recess 47 may be generally U-shaped when viewed from the distal end of the housing 40, with a proximal end of the recess 47 transitioning into the distal end of the bore 46, and with a distal end of the recess 47 opening from the distal face of the housing 40. The housing 40 may further define a slot 48 in communication with a base of the recess 47 and generally aligned therewith to accommodate components of the first actuator 100, and a bore 49 connecting a proximal face of the slot 48 to a proximal face of the housing 40.

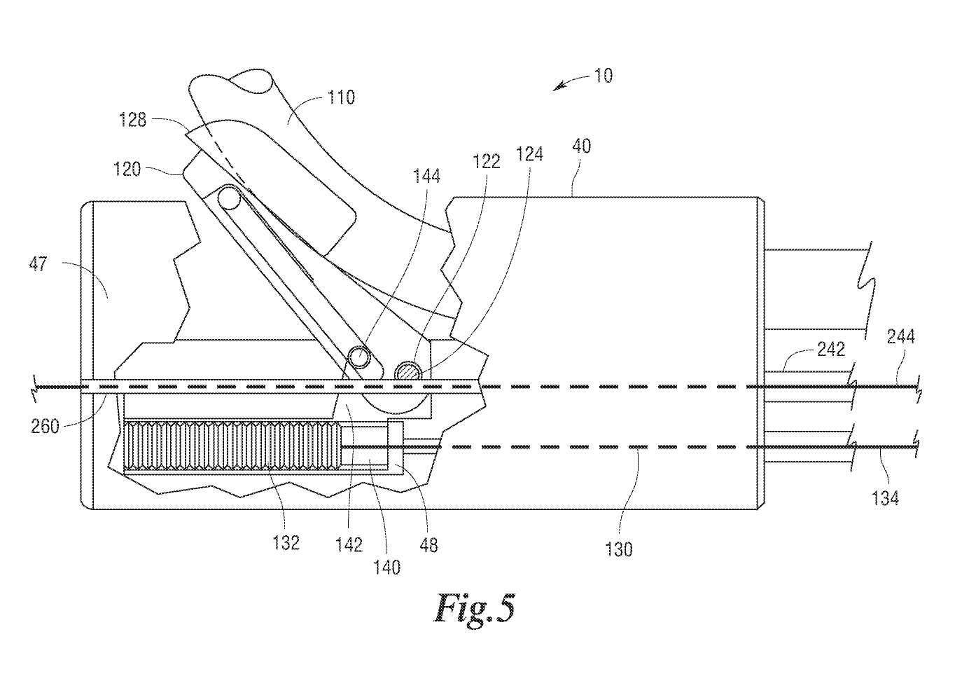

FIG. 5 illustrates a side view of the housing 60 with components of the first actuator 100 installed in the recess 47 and the slot 48. The first actuator 100 may comprise a pivot arm 120 having a proximal end pivotally attached to the housing 40 adjacent a proximal end of the slot 48. In one embodiment, pivotal cooperation between the pivot arm 120 and the housing 40 is accomplished using pivot pins 122 formed on opposing lateral surfaces of the proximal end of the pivot arm 120 that are cooperatively engaged by corresponding pivot recesses 124 defined by opposing lateral surfaces of the proximal end of the slot 48. Accordingly, the pivot arm 120 is pivotable between a lowered, non-deployed position in which the pivot arm 120 is predominantly or entirely contained within the recess 47, and an elevated, deployed position (as shown in FIG. 5) in which at least a distal portion of the pivot arm 120 is pivotably elevated to extend from the recess 47, thereby flexing the distal end of the endoscopic instrument to alter its position.

In certain embodiments, the first actuator 100 may comprise a drive shaft 130 having a distal end 132 disposed in and extending through the slot 48, with the distal end 132 coupled to the pivot arm 120 via a linkage 140 that is slidably disposed in the slot 48. As shown in FIG. 5, at least a portion of the distal end 132 of the drive shaft 130 contained within the slot 48 may be threaded. The linkage 140 may define a bore adapted to threadably receive the distal end 132 of the drive shaft 130. In this way, rotation of the distal end 132 of the drive shaft 130 may be employed to cause translation of the linkage 140 along a length of the slot 48. For example, rotation of the distal end 132 of the drive shaft 130 in a clockwise direction (e.g., as viewed from the proximal end of the inner sheath assembly 10 may cause translation of the linkage 140 in a proximal direction relative to the slot 48, while rotation of the distal end 132 of the drive shaft 130 in an opposite direction may cause the linkage 140 to translate in a distal direction relative to the slot 48. Rotation of the distal end 132 of the drive shaft 130 in this manner may be accomplished by rotating a proximal end 134 of the drive shaft 130 that proximally extends from the bore 49 and through the inner sheath 20. The proximal end 134 of the drive shaft 130 may be connected to a control device (e.g., a motor, a manually rotatable knob) (not shown) for suitably controlling the rotational position of the proximal end 134, and thus, the translatory position of the linkage 140 relative to the slot 48. In certain embodiments, at least a portion of the proximal end 134 of the drive shaft 130 (e.g., a portion of the drive shaft 130 extending through the inner sheath 20) may be rotatably housed within a flexible sleeve.

As further shown in the embodiment of FIG. 5, the pivot arm 120 may include a track 126 in the form of an elongate slot that is defined by lateral surfaces of the pivot arm 110 and that is slidably engaged by a pin 144 formed on an upwardly-extending arm 144 of the linkage 140. The configuration of the slot 126 may be such that when the linkage 140 is translated into its distal-most position relative to the slot 48 (e.g., by suitable rotation of the drive shaft 130), the resulting sliding engagement of the slot 126 by the pin 144 causes the pivot arm 120 to assume its lowered, non-deployed position. Conversely, as the linkage 140 is translated from its distal-most position in a proximal direction, the resulting sliding engagement of the slot 126 by the pin 144 causes the progressive elevation of the pivot arm 120, with the elevated, fully-deployed position of the pivot arm 120 corresponding to the proximal-most position of the linkage 140 relative to the slot 48. In this way, rotation of the drive shaft 130 may be used to selectively adjust the position of the pivot arm 120 between its lowered and elevated positions.

In certain embodiments, the distal end of the pivot arm 120 may comprise a guide surface 128 for slidably contacting a distal end of an endoscopic instrument introduced through the bore 46 via the working channel 26 in order to effectively transfer pivotal movement of the pivot arm 120 to the distal end of the endoscopic instrument. As shown in FIG. 2, for example, the guide surface 128 may be trough-shaped and comprise a curvature generally matching a curvature of an outer surface of the endoscopic instrument. In this way, the guide surface 128 may conform to a degree to the outer surface of the endoscopic instrument such that the endoscopic instrument is laterally retained on the guide surface 128 while permitting sliding contact of the endoscopic instrument with the guide surface 128 in the distal and proximal directions. In certain embodiments, the guide surface 128 may comprise a lubricious coating (e.g., a biocompatible Teflon® coating) to reduce frictional forces between the guide surface 128 and the endoscopic instrument.

It will be appreciated that translatory control of the linkage 140 may be achieved in a number of ways that do not require a rotatable drive shaft 130. In one embodiment, for example, the first actuator 100 may instead include a control cable assembly (not shown) comprising a flexible guide and a control member slidably disposed therein. A distal end of the flexible guide may be received by and retained within a proximal portion the bore 49 of the housing 40, with a distal end of the control member extending from the distal end of the flexible guide and through a distal portion of the bore 49 to attach to the linkage 140. The flexible guide may proximally extend through a length of the inner sheath 20 and comprise a proximal end attached to, for example, a handle coupled to the inner sheath 20. A distal end of the control member may extend from the proximal end of flexible guide to attach to a suitable mechanical or electromechanical actuator (e.g., a lever actuator, a knob actuator, a trigger actuator, a bar clamp actuator, a syringe grip actuator, a solenoid actuator, a motor actuator) for controllably translating the control member within the guide, thus causing translation of the linkage 140 and concomitant pivotal movement of the pivot arm 120.

In addition to or as an alternative to the use of an active (e.g., movable) actuator (e.g., first actuator 100) to selectively position the distal end of an endoscopic instrument introduced through the working channel 46, embodiments of the inner sheath assembly 10 may comprise one or more passive (e.g., stationary) guide surfaces to control distal end position by virtue of movement of the distal end relative to the passive guide surface(s). In certain cases, use of passive guide surfaces may be preferable to active actuators in terms of reduced size, ease of manufacture, reduced cost, and/or for addition of components/elements in a space that would otherwise be occupied by components/elements of an active actuator.

Embodiments of the inner sheath assembly 10 may further comprise one or more second actuators to controllably manipulate the articulation joints 60, 70. In one such embodiment, for example, each articulation joint 60, 70 may be manipulated by a corresponding second actuator 230, 240, with each actuator 230, 240 respectively comprising a flexible guide 232, 242 and a corresponding control member 234, 244 slidably disposed therein. As shown in FIGS. 3-5, the housing 40 may define bores 250, 260 extending longitudinally through the housing 40 between the proximal and distal faces thereof for respectively accommodating distal portions of the second actuators 320a, 320b. Each bore 250, 260 may define a first diameter to receive and retain a distal portion of the corresponding flexible guide 232, 242, and a second diameter distal the first diameter to receive a distal portion of the corresponding control member 234, 244. Distal portions of the control members 234, 244 passed through bores 250, 260 of the housing 40 may be slidably received through corresponding auxiliary bores 62, 72 defined by the sidewalls of the articulation joints 80a, 80b, with the auxiliary bores 62, 72 being respectively aligned with the bores 250, 260 when the articulation joints 60, 70 are in an un-articulated state. Distal tips of the control members 234, 244 may respectively attach to the articulation joints 60, 70 adjacent the distal ends of their corresponding auxiliary bores 64, 66. In this way, each control member 234, 244 may be slidably translated through its respective flexible guide 232, 242, bore 250, 260 and auxiliary bore 62, 64 to controllably manipulate the corresponding articulation joint 60, 70. In certain embodiments, for example, independent translation of the control members 234, 244 may be accomplished using a suitable mechanical or electromechanical actuator (e.g., a lever actuator, a knob actuator, a trigger actuator, a bar clamp actuator, a syringe grip actuator, a solenoid actuator, a motor actuator) (not shown) attached to the proximal end of each control member 234, 244 adjacent a handle coupled to the inner sheath 40.

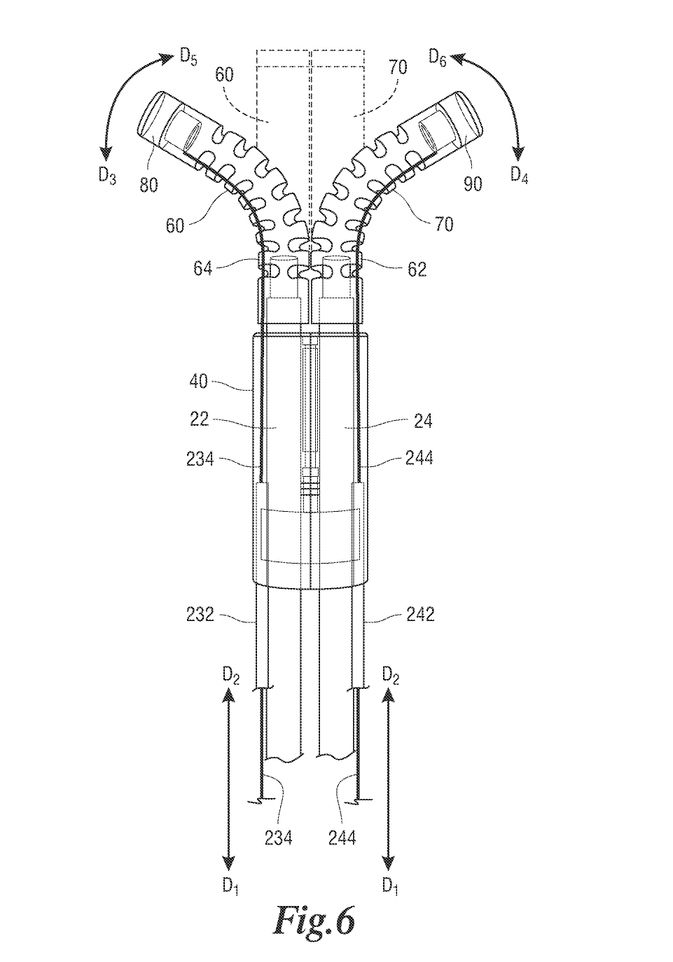

FIG. 6 is a bottom view of a distal portion of the inner sheath assembly 10 illustrating deflection of the articulation joints 80a, 80b in response to translation of their corresponding control members 234, 244. As shown, both control members 234, 244 have been translated equal distances in the proximal direction D₁, thus causing the articulation joints 60, 70 to be equally deflected in directions D₃ and D₄, respectively. Subsequent translation of the control members 234, 24 in the distal direction D₂ will reduce the degree of deflection by causing the articulation joints 60, 70 to move in directions D₅ and D₆, respectively, such that the articulation joints 60, 70 eventually assume their un-deflected positions (indicated in FIG. 6 by the phantom outline of the articulation joints 60, 70). Although not illustrated in FIG. 6, it will be appreciated that the articulation joints 60, 70 may be deflected in the same direction by translating the control members 234, 244 in opposite directions. For example, translating control member 234 in the proximal direction D₁ while simultaneously translating control member 244 in the distal direction D₂ will result in the deflection of the articulation joints 60, 70 in the direction D₃. Conversely, translating control member 234 in the distal direction D₂ while simultaneously translating control member 244 in the proximal direction D₁ will result in the deflection of the articulation joints 60, 70 in the direction D₄.

Those of ordinary skill in the art may appreciate that the inner sheath assembly 10 is not a particularly well-suited for initial insertion through a patient's natural orifice. For example, due to the generally protruding orientations of the articulation joints 60, 70 as well as the endoscopic tool 110, the inner sheath assembly 10 is challenging to insert through fragile organs such as the esophagus and the like without damaging tissue. The various embodiments of the present invention may solve some of those shortcomings.

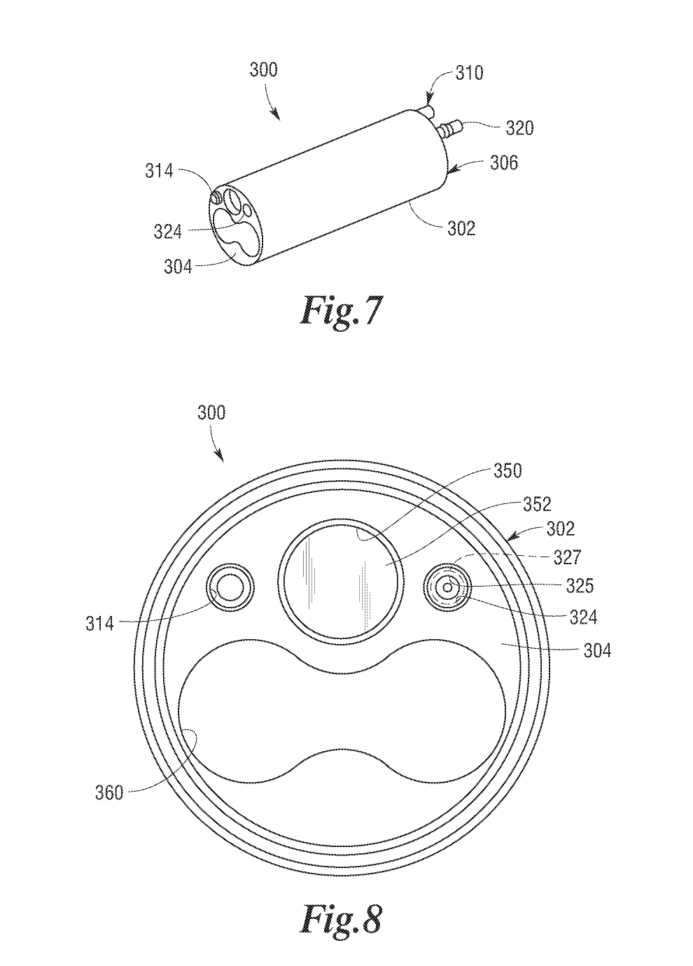

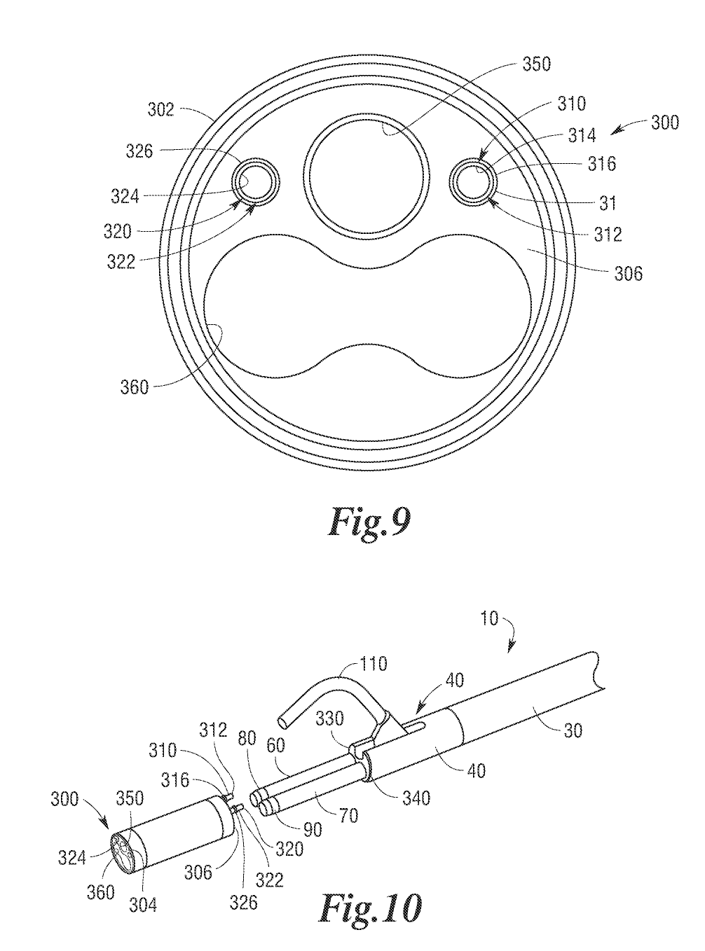

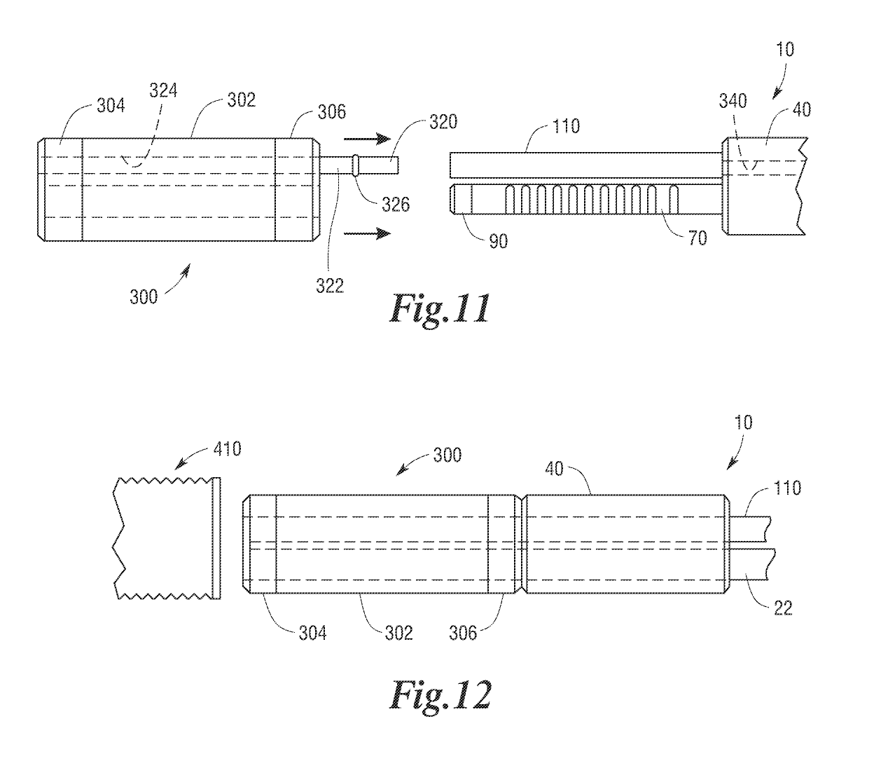

In particular and with reference to FIGS. 7-12, there is shown an intralumenal accessory tip assembly 300 that is configured for removable attachment to the distal end of the housing 40. In various embodiments, the tip assembly 300 may have a central body portion 302 that has a distal end cap 304 and a proximal end cap 306. In various embodiments, the central body portion 302 may be substantially cylindrical in shape. The body portion 302 and end caps 304, 306 may be fabricated from a suitable biocompatible metal, rubber (or similar pliable material), or plastic material. The end caps 304 and 306 preferably have smooth round edges and may be slightly chamfered to avoid tissue damage as the tip assembly 300 is passed through the body. The tip assembly 300 may further include a first attachment member or conduit 310 that is attached or otherwise formed with the body portion 302 such that a first attachment portion 312 protrudes in a proximal direction from the proximal end cap 304. The first attachment member or conduit 310 may be substantially hollow and define a first tip lumen 314 that extends through the body portion 302. In addition, a second attachment member or conduit 320 is attached or otherwise formed with the body portion 302 such that a second attachment portion 322 protrudes in the proximal direction from the proximal end cap 304. The second conduit 230 may be substantially hollow and define a second tip lumen 324 that extends through the body portion 302 of the tip assembly 300.

As can be seen in FIG. 10, the first attachment member 310 is adapted to be received in a corresponding first attachment lumen 330 that may extend completely through the housing 40 of the inner sheath assembly 10. Likewise, the second attachment member 320 is adapted to be received in a corresponding second attachment lumen 340 in the housing 40. In various embodiments, at least one first O-ring 316 may be journaled on the attachment portion 312 such that when first attachment portion 312 is inserted into the first attachment lumen 330 in the housing 40, a slidable frictional fit is established therebetween. It will also be appreciated that the first O-ring 316 may also establish a substantially fluid-tight seal between the first attachment portion 312 and the first attachment lumen 333. Likewise, at least one second O-ring 326 may be journaled on the second attachment portion 322 such that when the second attachment portion 322 is inserted into the second attachment lumen 340, another slidable frictional fit is established therebetween. It will also be appreciated that the second O-ring 326 may also establish a substantially fluid-tight seal between the second attachment portion 322 and the second attachment lumen 340. Thus, when the first and second attachment portions 312, 322 are inserted into the first and second lumens 330, 340, respectively, the tip assembly 300 is removably attached to the housing 40 of the inner sheath assembly 10. In various embodiments, the first attachment lumen 330 and the first tip lumen 314 may form a passage for receiving tools therethrough. Likewise, the second attachment lumen 340 and the second tip lumen 324 may form a passage for receiving tools therethrough.

In various embodiments, for example, a nozzle 325 may be provided in the second tip lumen 324 that is in fluid communication with a fluid channel 327 extending through the inner sheath assembly 10. See FIG. 8. Such arrangement may provide the clinician with a means to spray water to clear organic material from the other tools/instruments (a camera lens, for example) inserted through the sheath assembly 10.

As can be seen in FIGS. 8-10, the tip accessory 300 further has a centrally disposed tool passage 350 formed therein to accommodate the endoscopic tool 110. In various embodiments, wherein the endoscopic tool comprises a camera, the distal end of the tool passage 350 may be provided with a lens or protective transparent cover plate 352 to prevent organic matter from collecting on the camera or other tool inserted tin the tool passage 350 as the tip assembly 300 and sheath 10 are inserted into the patient. In other embodiments, the lens 352 may be omitted. As can also be seen in FIGS. 8-10, the tip assembly 300 may be formed with a tube-receiving passage 360 shaped to accommodate the articulation joints 60, 70 when the joints 60, are essentially axially aligned (not articulated) with respect to the inner sheath 20. The tip assembly 300 may be sized such that when attached to the housing 40 of the sheath assembly 10, the articulation joints 60, 70 do not protrude out beyond the distal end cap 304. See FIG. 12.

While the embodiment described above, employs two attachment members 310, 320, one attachment member or more than two attachment members may be employed to removably attach the tip assembly 300 to the housing 40 of the inner sheath assembly 10. Other variations may include attachment members that are not hollow and do not form lumens through which tools may be inserted. Still other variations for affixing the tip assembly to the housing 40 may be employed. For example, the tip assembly 300 may be removably attached to the housing 40 by removable fasteners such as screws. In other embodiments, the attachment members 310, 320 protrude from the distal end of the housing 40 for insertion into corresponding lumens provided in the proximal end cap 306 of the tip assembly 300. In other embodiments one of the attachment members 310, 320 may protrude from the proximal end cap 306 to be received in a lumen in the housing 40 and the other attachment member 310, 320 may protrude from the distal end of the housing 40 to be received in a lumen in the proximal end cap 306.

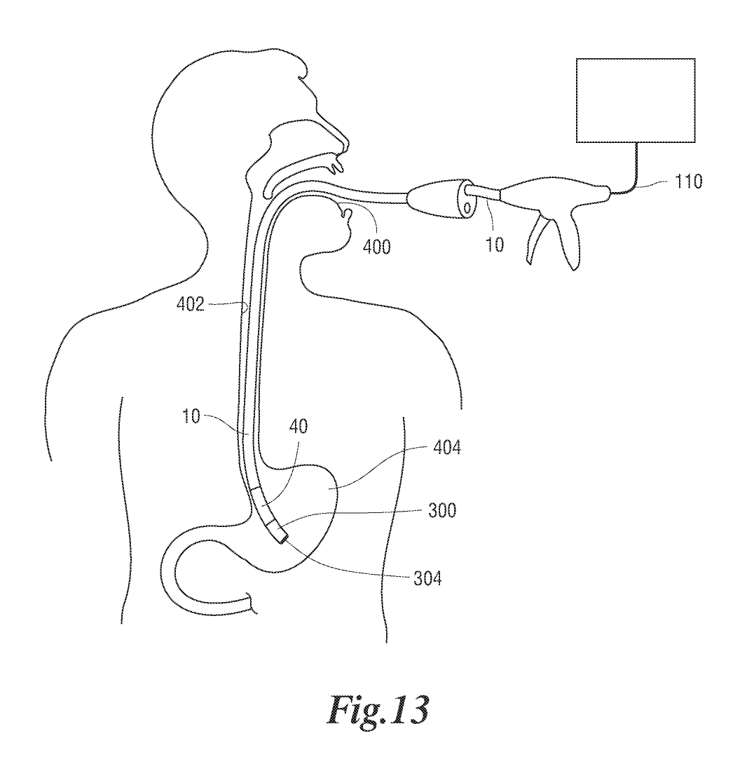

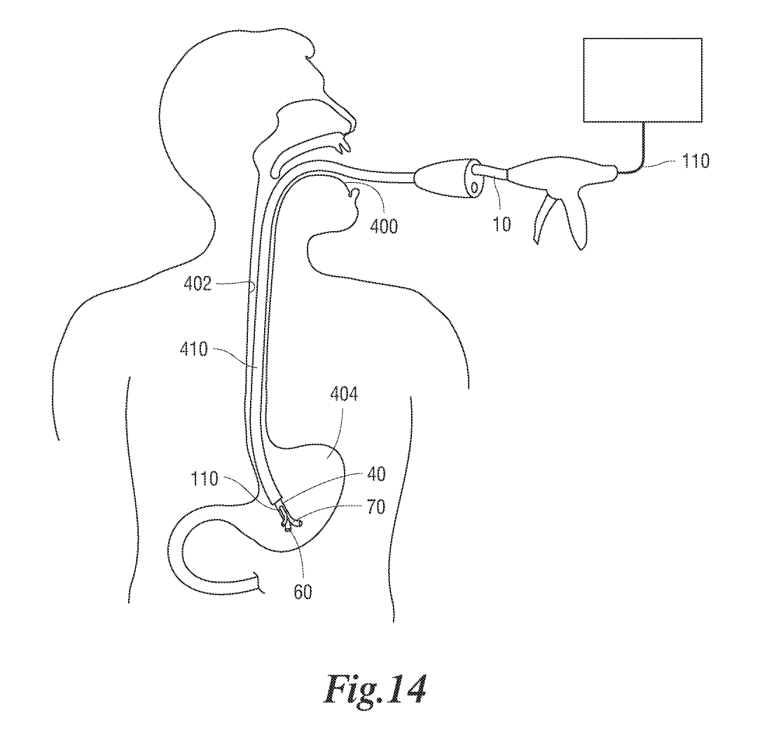

One method of using the tip assembly 300 will now be described with reference to FIGS. 13 and 14. For example, the tip assembly 300 may be attached to the housing 40 of the sheath assembly 10 and an overtube assembly 410 may be positioned over the sheath assembly 10 prior to insertion of the device into the patient. The overtube assembly 410 may be positioned such that the distal tip of the sheath assembly 10 is either recessed, flush, or exposed relative to the tip of the overtube assembly 410. After the tip assembly 300 has been attached to the housing 40, the clinician can insert the tip assembly 300, overtube assembly 410, and inner sheath assembly 10 into the patient. For example, the combined assemblies 300, 410, 10 may be inserted through a patient's mouth 400 and down the patient's esophagus 402 and into the stomach 404. See FIG. 13. The smooth distal end cap 304 of the tip assembly 300 minimizes the likelihood of tissue damage during the insertion process. After the clinician has determined that the tip assembly 300 has been inserted to a desired position (for example, using the camera 110), the clinician may withdraw the inner sheath assembly 10 from the overtube 400. The tip assembly 300 may then be removed and the inner sheath assembly 10 reinserted into the patient through the overtube 400 to commence the operation. See FIG. 14.

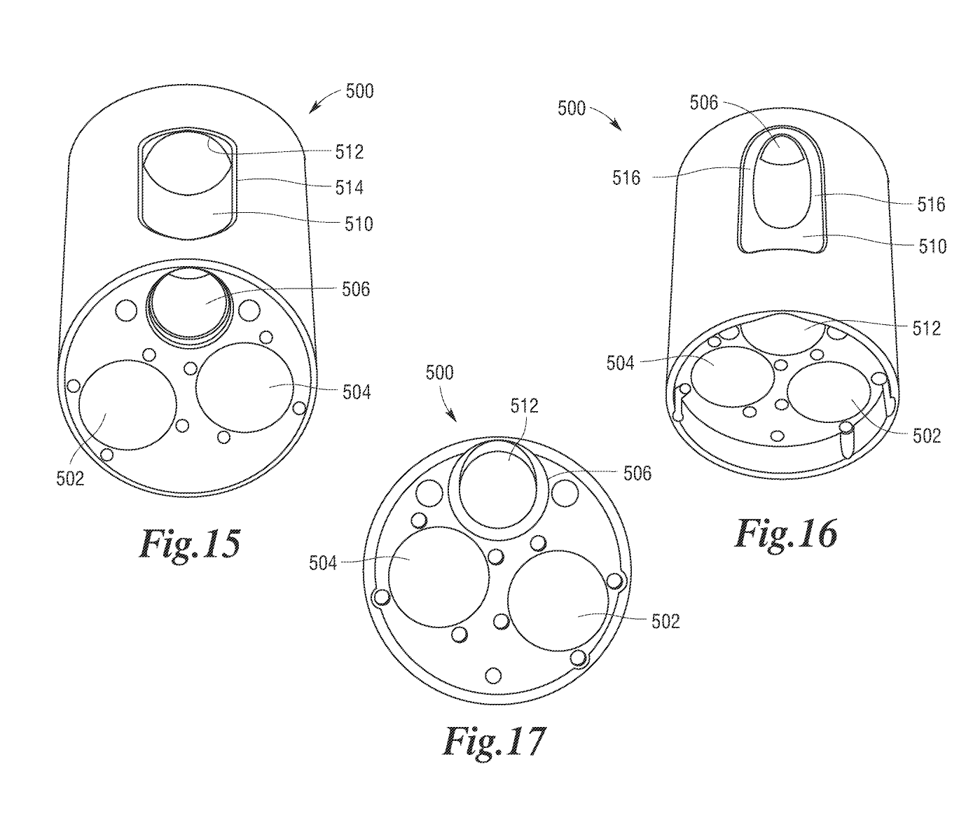

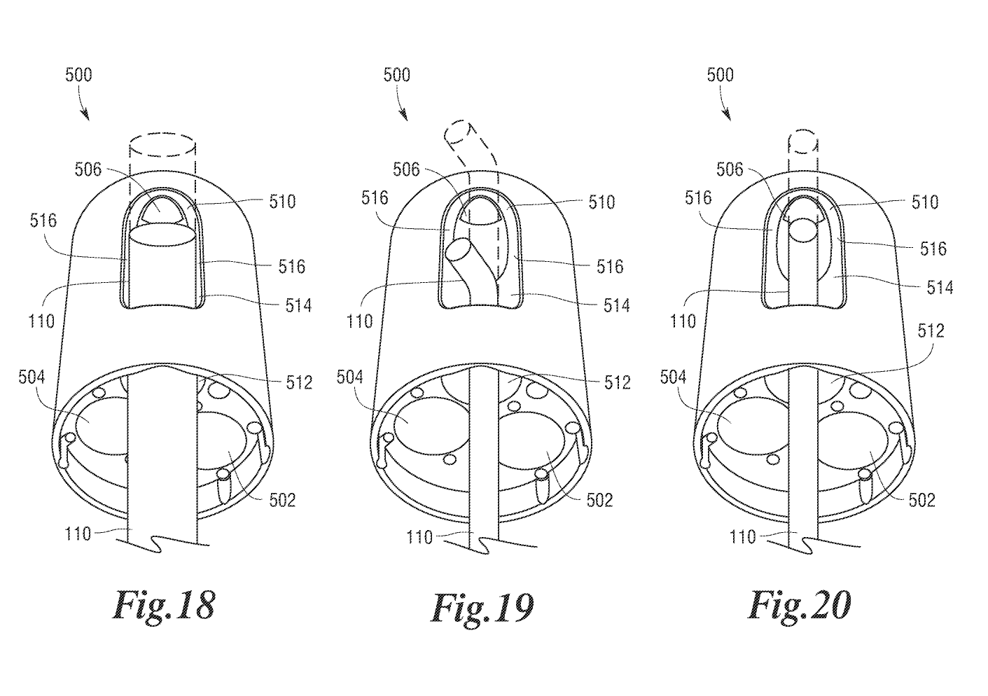

FIGS. 15-17 illustrate front perspective and rear perspective views, respectively, of a housing 500 that may be used in connection with another embodiment of an inner sheath assembly. FIG. 15 illustrates a rear view of the housing 500. The housing 500 may be similar in certain respects to the housing 40 and define, for example, bores 502, 504, 506 that extend longitudinally and at least partially through the housing 500 and receive the distal ends of the working channels 22, 24, 26, respectively, at least partially therethrough. In FIGS. 13-15, the working channels 22, 24, 26 have been omitted for the sake of clarity. The housing 500 may further define a recess 510 that is in communication with the bore 506 and generally aligned therewith to receive and guide a distal end of an endoscopic instrument 110 introduced through the bore 506 via the working channel 26. As shown, the housing 500 may define separate openings connected to the recess 510 from which the distal end of the endoscopic instrument may exit the housing 500 subsequent to its introduction into the recess 510 via the bore 46. For example, a bore 512 may be defined by the housing 500 to provide a transition from the distal end of the recess 510 through the distal face of the housing 500, and an opening 514 may be defined by the housing 500 such that a portion of the recess 510 is exposed through a lateral surface of the housing 500. As shown in FIG. 14, a distal wall of the recess 510 may define a proximal opening of the bore 512 and comprise a curved surface that is continuous with base and lateral surfaces of the recess 510 and that slopes upward relative to the base surface of the recess 510 in the distal direction. The distal wall of the recess 510 thus defines a ramped guide surface 516 disposed adjacent the proximal opening of the bore 512 to slidably engage and position the distal end of an endoscopic instrument as the distal end is moved in the distal direction relative to the ramped guide surface 516. In certain embodiments, for example, a width of a distal tip portion of the endoscopic instrument (e.g., a distal tip portion of camera 110) may be equal to or slightly smaller than a width of the ramped guide surface 514, but larger than a width of the proximal opening of the bore 512, such that the distal tip portion is not passable through the bore 512. Accordingly, as shown in FIG. 16, as the distal tip portion of the endoscopic instrument 110 is advanced through the recess 510, the distal tip portion is slidably engaged by the ramped guide surface 516. Continued advancement of the distal tip portion (indicated in FIG. 16 by phantom outline) through the recess 510 causes the distal tip portion to follow the upward-sloping contour of the ramped guide surface 516 and eventually emerge from the recess 510 via the opening 514. In other embodiments, the width of distal tip portion may be smaller than a width of the proximal opening of the bore 512 such that passage of the distal tip portion through either the bore 512 or the opening 514 is possible. In such embodiments and as shown in FIG. 17, for example, the distal tip portion may be suitably articulated within the recess 510 (e.g., using an actuator of the endoscopic instrument 110) such that at least a portion of the ramped guide surface 514 slidably engages the distal tip portion. Continued advancement of the articulated distal tip portion through the recess 510 (indicated in FIG. 17 by phantom outline) causes the distal tip portion to follow the upward-sloping contour of the ramped guide surface 514 and eventually emerge from the recess 510 via the opening 514. Alternatively, as shown in FIG. 18, the distal tip portion may be advanced through the recess 211 in an unarticulated state such that distal tip portion is not slidably engaged by the ramped guide surface 214. In this case, continued advancement of the distal tip portion through the recess 510 (indicated in FIG. 18 by phantom outline) results in emergence of distal tip portion from the distal face of the housing 500 via the bore 512.

The tip assembly 300 as described above may also be employed in connection with the housing 500. For example, a first attachment lumen 520 and a second attachment lumen 530 may be provided in the housing 500 for receiving the first and second attachment members 310 and 320, respectively therein. Those of ordinary skill in the art will understand that the camera or other endoscopic tool 110 must be oriented in the position shown in FIG. 18 to enable the tool 110 to be inserted into the centrally disposed tool passage 350. The tip assembly 300 and housing 500 may otherwise be installed an employed in the various manners described above.

While the illustrative embodiments have been described in considerable detail, it is not the intention of the applicant to restrict or in any way limit the scope of the appended claims to such detail. Additional advantages and modifications may readily appear to those skilled in the art. The intralumenal tip accessory embodiments serve to avoid tissue damage that might otherwise be caused when inserting inner sheath assemblies that have tools and access tubes protruding therefrom. It will be understood that the intralumenal accessory tip embodiments of the present invention may be effectively employed with a variety of different inner sheath configurations without departing from the spirit and scope of the present invention. Those of ordinary skill in the art will also understand that such inner sheath arrangements may also be used in connection with a variety of different camera arrangements. For example, to further enhance the surgical experience, a camera may be employed that has zoom capability (either digital or optical). Such a camera may be employed to mimic laparoscopic capabilities associated with moving a laparoscope during laparoscopic surgery for example, to provide a stadium view and a detailed view of the tissue as required by the clinician. The various accessory tip embodiments of the present invention may employ a cover lens or other transparent protective cover that facilitates viewing by the camera, yet prevents organic material from fouling the camera during the insertion process. While the various embodiments of the present invention have been described herein in connection with the performance of surgical procedures through a natural orifice of the patient, those of ordinary skill in the art will appreciate that the various embodiments of the present invention may also be effectively introduced through an incision in the patient.

While the embodiments have been described, it should be apparent, however, that various modifications, alterations and adaptations to the embodiments may occur to persons skilled in the art with the attainment of some or all of the advantages of the invention. For example, according to various embodiments, a single component may be replaced by multiple components, and multiple components may be replaced by a single component, to perform a given function or functions. This application is therefore intended to cover all such modifications, alterations and adaptations without departing from the scope and spirit of the disclosed invention as defined by the appended claims.

The devices disclosed herein can be designed to be disposed of after a single use, or they can be designed to be used multiple times. In either case, however, the device can be reconditioned for reuse after at least one use. Reconditioning can include a combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility.

Those of ordinary skill in the art will appreciate that the devices disclosed herein may be provided in a kit that may, for example, be directed to a particular surgical procedure. For example, a kit may include an accessory tip 300 in combination with an inner sheath assembly 10 and an overtube assembly 410 that may be particularly well-suited to accommodate those endoscopic tools likely to be employed during a particular surgical procedure. In other embodiments, the kit may include a plurality of accessory tips 300 that each are configured to be used in connection with a different inner sheath assembly included therewith. Such kit arrangements provide the clinician with the added flexibility to select the appropriate inner sheath assembly 10 for a particular procedure and to affix the appropriate accessory tip to facilitate insertion of the inner sheath assembly into the patient without an overtube first being installed.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An intralumenal accessory tip for attachment to an inner sheath assembly having a housing and at least one flexible access tube and an endoscopic tool protruding from a distal end of the housing, said intralumenal accessory tip comprising:
a body portion having a distal end and a proximal end, said proximal end configured for removable attachment to the housing;
a tool-receiving passage corresponding to the endoscopic tool and sized to receive the endoscopic tool therein when said proximal end of said body portion is attached to the distal end of the housing such that no part of the endoscopic tool protrudes beyond said distal end of said body portion; and
a tube-receiving passage sized to receive at least one of the at least one flexible access tubes therein when said proximal end of said body portion is attached to the distal end of the housing such that no portion of the at least one flexible access tube protrudes beyond said distal end of said body portion.

2. The intralumenal accessory tip of claim 1 wherein said proximal end of said body portion is attached to the distal end of the housing by at least one attachment member protruding from said proximal end of said body portion.

3. The intralumenal accessory tip of claim 2 wherein the housing has first and second attachment lumens extending therethrough and wherein said at least one attachment member comprises:
a first attachment conduit protruding from said proximal end of said body portion and sized to be retainingly inserted into the first attachment lumen in the housing; and
a second attachment conduit protruding from said proximal end of said body portion and sized to be retainingly inserted into the second attachment lumen in the housing.

4. The intralumenal accessory tip of claim 3 further comprising:
at least one first retainer on said first attachment conduit; and
at least one second retainer on said second attachment conduit.

5. The intralumenal accessory tip of claim 4 wherein at least one of said at least one first retainer comprises a first O-ring and wherein at least one of said at least one second retainer comprises a second O-ring.

6. The intralumenal accessory tip of claim 1 wherein said tube-receiving passage is configured to receive two flexible access tubes therein.

7. The intralumenal accessory tip of claim 1 further comprising a viewing lens or protective transparent cover plate corresponding to said tool-receiving passage.

8. The intralumenal accessory tip of claim 1 further comprising a nozzle in communication with a fluid channel in the inner sheath assembly.

9. The intralumenal accessory tip of claim 1 wherein said body portion is substantially cylindrically shaped and wherein a chamfered corner is formed around a circumference of said distal end of said body portion.

10. An intralumenal accessory tip for attachment to an inner sheath assembly having a housing and two articulatable access tubes protruding therefrom and an endoscopic camera having a selectively movable end portion protruding from a distal end of the housing, said intralumenal accessory tip comprising:
a body portion having a distal end and a proximal end;
means for removably attaching said proximal end of said body portion to the distal end of the housing;
tube access means in said body portion for accommodating the two articulatable access tubes when said body portion is attached to the distal end of the housing; and
tool access means in said body portion for accommodating the endoscopic camera therein when said body portion is attached to the distal end of the housing.

11. The intralumenal accessory tip of claim 10 wherein said means for removably attaching form at least one tool-receiving lumen extending through said body portion.

12. The intralumenal accessory tip of claim 11 wherein the housing has an attachment lumen extending therethrough that corresponds to one of said tool-receiving lumens in said body portion.

13. The intralumenal accessory tip of claim 12 further comprising means for establishing a substantially fluid-tight seal between said tool receiving lumen and said corresponding attachment lumen when said body portion is attached to the housing.

14. The intralumenal accessory tip of claim 10 wherein said means for removably attaching comprises:
a first hollow conduit extending from said proximal end of said body portion and sized to be removably received within a corresponding first attachment lumen formed in the housing;
first means for removably retaining said first hollow conduit in the first attachment lumen;
a second hollow conduit extending from said proximal end of said body portion and sized to be removably received within a corresponding second attachment lumen formed in the housing; and
second means for removably retaining said second hollow conduit in the second attachment lumen.

15. The intralumenal accessory tip of claim 14 wherein said first means for removably retaining also establishes a first substantially fluid-tight seal between said first hollow conduit and the first attachment lumen when said first hollow conduit is inserted therein and wherein said second means for removably retaining establishes a second substantially fluid-tight seal between said second hollow conduit and the second attachment lumen when said second hollow conduit is inserted therein.

16. The intralumenal accessory tip of claim 14 wherein said first means for removably retaining establishes a sliding frictional fit between first hollow conduit and the first attachment lumen when said first hollow conduit is inserted therein and wherein said second means for removably retaining establishes another sliding frictional fit between said second hollow conduit and the second attachment lumen when said second hollow conduit is inserted therein.

17. The intralumenal accessory tip of claim 10 further comprising means for preventing organic matter from accumulating in said tool access means.

18. The intralumenal accessory tip of claim 10 further comprising means for clearing organic material from a protective cover plate or lens protecting said tool accessory means.

19. A surgical method comprising:
attaching an intralumenal accessory tip onto a distal end of an internal sheath assembly such that all of any an portions of accessory tubes and endoscopic tools protruding from the distal end of the internal sheath assembly are received within the intralumenal accessory tip and do not protrude beyond a distal end of the intralumenal accessory tip;
inserting an overtube over the internal sheath assembly and intralumenal accessory tip such that a distal end of the overtube is located in a desired position relative to the intralumenal accessory tip;
inserting the intralumenal accessory tip, a portion of internal sheath assembly, and the overtube into a patient such that the intralumenal accessory tip is located in a desired area;
withdrawing the internal sheath assembly and intralumenal accessory tip out through the proximal end of the overtube while retaining the overtube in place;
removing the intralumenal accessory tip from the distal end of the internal sheath assembly; and
reinserting the internal sheath assembly into the overtube to the desired area.

20. The surgical method of claim 19 wherein said inserting the intralumenal accessory tip and a portion of the internal sheath assembly and the overtube into a patient comprises inserting the intralumenal accessory tip, a portion of internal sheath assembly and overtube through a natural orifice of the patient.

21. The surgical method of claim 19 wherein said inserting the intralumenal accessory tip and a portion of the internal sheath assembly and the overtube into a patient comprises inserting the intralumenal accessory tip and a portion of internal sheath assembly and overtube through a surgical incision in the patient.

22. The surgical method of claim 19 wherein one of the endoscopic tools on the internal sheath assembly comprises a camera and wherein said surgical method further comprises employing said camera during said inserting of the intralumenal accessory tip and a portion of internal sheath assembly into the patient.

23. The surgical method of claim 19 further comprising passing at least one surgical tool through the internal sheath assembly and the intralumenal accessory tip prior to said withdrawing.
